(19) United States
(12) Patent Application Publication
(10) Pub. No.: US 2004/0052828 A1
(43) Pub. Date: Mar. 18, 2004

(54) Consumable product including consumable component and alpha-keto enamine derivatives

(76) Inventors: Thomas Hofmann, Muenster-Roxel (DE); Harald Ottinger, Freiburg (DE); Oliver Frank, Munster (DE); Tomislav Soldo, Memmingen (DE); Christoph Cerny, La Conversion (CH); Fabien Robert, Divonne les Bains (FR); Imre Blank, Savigny (CH)

Correspondence Address: WINSTON & STRAWN, PATENT DEPARTMENT, 1400 L STREET, N.W., WASHINGTON, DC, 20005-3502, US

(21) Appl. No.: 10/448,306

(22) Filed: May 30, 2003

Related U.S. Application Data
(63) Continuation-in-part of application No. 09/863,970, filed on May 23, 2001, now Pat. No. 6,592,884.

(30) Foreign Application Priority Data
May 23, 2000 (EP) 00110886

(51) Int. Cl. A61K 31/445; A61K 7/00; A61K 31/40; A61K 31/12

(52) U.S. Cl. 424/401; 514/408; 514/690; 514/317

(57) ABSTRACT

The present invention relates to a consumable product that includes a consumable component and a cooling component present in an amount sufficient to provide a cooling sensation to a consumer, which cooling component includes one or more compounds each having the general formula:

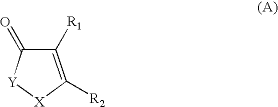

wherein R₁ is selected from the group consisting of N-Pyrrolidinyl, N-Pyridinyl, N-(aminodiethyl), N-(2-carboxy-pyrrolidinyl), piperidinyl, or N-(2-Methoxycarbonyl-pyrrolidinyl); R₂ is selected from the group consisting of hydrogen or methyl; X is selected from the group consisting of methylene, ethylidene, 1-Propylidene, or oxy radical; and Y is selected from the group consisting of methylene, ethylidene, 1-propylidene, oxy radical, ethan-1,2-diyl, ethen-1,2-diyl, propan-1,2-diyl, or ethan-1-oxy-1-yl, provided that when R₁ is N-pyrrolidinyl, X is methylene, and Y is ethylidene then R₂ cannot be hydrogen, and provided that when R₁ is N-pyrrolidinyl, and X and Y are each methylene, then R₂ cannot be methyl. In particular, the consumable product can be a food product, perfume, cosmetic, or pharmaceutical.

### CROSS-REFERENCE TO RELATED APPLICATIONS

This application is a continuation-in-part of U.S. patent application Ser. No. 09/863,970, filed May 23, 2001, now allowed, the contents of which is hereby incorporated herein by express reference thereto.

### FIELD OF THE INVENTION

The present invention relates to consumable products containing a consumable component and cooling component that includes one or more alpha-keto enamine derivatives, as well as food products, perfumes, pharmaceuticals, and cosmetics formed from the consumable products.

### BACKGROUND OF THE INVENTION

The Maillard reaction of L-proline with reducing monosaccharides has been extensively studied during the last two decades in order to gain insights into the formation of volatiles during thermal processing of cereal products (Tressl et al., J. Agric. Food Chem. 1985a, 33, 919-923; Tressl et al. J. Agric. Food Chem. 1985b, 33, 924-928; Tressl et al. J. Agric. Food Chem. 1985c, 33, 1132-1137; Helak et al. J. Agric. Food Chem. 1989a, 37, 400-404; Helak et al. J. Agric. Food Chem. 1989b, 37, 405-410; Huyghues-Despointes et al. J. Agric. Food Chem. 1994, 42, 2519-2524).

By application of the GC/olfactometry techniques such as Charm analysis (Roberts, D. D., Acree, T. In Thermally Generated Flavors; Parliment T. H., Morello M. J., McGorrin R., Eds.; ACS, Washington D.C., 1994, 71-79) or aroma extract dilution analysis (AEDA)(Hofmann, T., Schieberle, P. J. Agric. Food Chem. 1998, 46, 2721-2726), the odor-active compounds could be successfully detected in solvent extracts of Maillard reaction systems composed of L-proline and reducing sugars. Amongst the volatiles detected, the popcorn-like smelling compounds 2-acetyl-1-pyrroline and 2-acetyltetrahydropyridine could be identified as the key contributors to the overall odor of thermally processed glucose/proline mixtures (Hofmann and Schieberle, J. Agric. Food Chem. 1998, 46, 2270-2277). Although the major part of the volatile reaction products formed during these roasting processes could be unequivocally shown by AEDA to have no odor activity, it cannot be excluded that some of these odorless compounds might evoke a certain taste sensation on the tongue such as, e.g., bitterness, heating or cooling. Consequently, the sensory attributes of such reaction products from reducing carbohydrates and proline were characterized.

By application of the recently developed taste dilution analysis (Hofmann, T. J. Agric. Food Chem. 1999, 47, 4763-4768) on HPLC fractions obtained from roasted glucose/proline mixtures two compounds could be detected, which showed an intense cooling effect on the tongue. These compounds were found to be formed in high concentrations when the hexose degradation product 2-hydroxy-3-methyl-2-cyclopenten-1-one was reacted in the presence of L-proline. After isolation by column chromatography both compounds could be obtained as pale-yellow oils with a purity of more than 99%. GC/MS and ID- and 2D-NMR spectroscopy led to the unequivocal identification of these cooling compounds as 5-methyl-2-(1-pyrrolidinyl)-2-cyclopenten-1-one (5-MPC) and 3-methyl-2-(1-pyrrolidinyl)-2-cyclopenten-1-one (3-MPC). 3-MPC 5-MPC

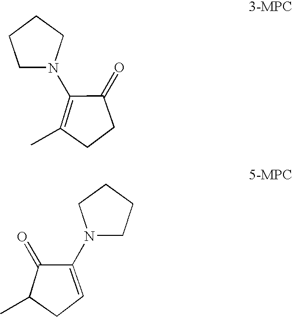

Although these compounds have been reported earlier by Tressl et al. (1985c) and Huyghues-Dispointes et al. (1994), these authors did not report on the cooling activity of these compounds when contacted with the tongue.

Besides 3-MPC and 5-MPC, we identified another cooling-active compound in the glucose/proline mixture, namely 2,5-dimethyl-4-(1-pyrrolidinyl)-3(2H)-furanone (DMPF).

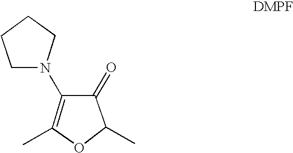

3-MPC as well as 5-MPC could easily be synthesised by heating 2-hydroxy-3-methyl-2-cyclopenten-1-one and pyrrolidinium acetate in ethanolic solution or by dry-heating of 2-hydroxy-3-methyl-2-cyclopenten-1-one in the presence of proline. In analogy, DMPF could be obtained by reacting 4-hydroxy-2,5-dimethyl-3(2H)-furanone in the presence of pyrrolidinium acetate in ethanol or in the presence of proline under dry-heating conditions, respectively.

Japanese Patent 7242661 discloses DMPF as flavoring compound to impart flavour to wheat flour foods. This patent concerns flavor emitted from such foods that were just heated. Hence the patent deals with DMPF as a flavour precursor. GB Patent No. 1096427 concerns certain cyclopentanone derivatives as interesting compounds for perfumery. Certain alpha-keto enamines are claimed, but they are mentioned only as intermediates for the synthesis of other compounds.

Thus, it is desired to obtain an improved cooling agent, particularly for use as an ingredient in foodstuffs or other consumable products.

### SUMMARY OF THE INVENTION

The invention encompasses a consumable product that includes a consumable component, and a cooling component present in an amount sufficient to provide a cooling sensation to a consumer, which component comprises one or more compounds each having the general formula:

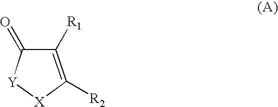

wherein R₁ is selected from the group consisting of N-Pyrrolidinyl, N-Pyridinyl, N-(aminodiethyl), N-(2-carboxy-pyrrolidinyl), piperidinyl, or N-(2-Methoxycarbonyl-pyrrolidinyl); R₂ is selected from the group consisting of hydrogen or methyl; X is selected from the group consisting of methylene, ethylidene, 1-Propylidene, or oxy radical; and Y is selected from the group consisting of methylene, ethylidene, 1-propylidene, oxy radical, ethan-1,2-diyl, ethen-1,2-diyl, propan-1,2-diyl, or ethan-1-oxy-1-yl, provided that when R₁ is N-pyrrolidinyl, X is methylene, and Y is ethylidene then R₂ cannot be hydrogen, and provided that when R₁ is N-pyrrolidinyl, and X and Y are each methylene, then R₂ cannot be methyl.

In one preferred embodiment, R₁ is N-pyrrolidinyl, R₂ is methyl, X is oxy radical and Y is methylene. In another preferred embodiment, R₁ is N-Pyrrolidinyl, R₂ is methyl, X is methylene and Y is oxy radical. In yet another preferred embodiment, R₁ is N-Pyrrolidinyl, R₂ is methyl, X is ethylidene and Y is oxy radical.

Preferred compounds in the cooling component include at least one of 3-Methyl-2-(1-pyrrolidinyl)-2-cyclopenten-1-one (3-MPC), 5-Methyl-2-(1-pyrrolidinyl)-2-cyclopenten-1-one (5-MPC), 3-Methyl-2-(1-piperidinyl)-2-cyclopenten-1-one (3-MPipC), 5-Methyl-2-(1-piperidinyl)-2-cyclopenten-1-one (5-MPipC), 3-Methyl-2-diethylamino-2-cyclopenten-1-one (3-MDeaC), 5-Methyl-2-diethylamino-2-cyclopenten-1-one (5-MDeaC), 3-Methyl-2-diethylamino-2-cyclopenten-1-one (3-MDeaC), 5-Methyl-2-diethylamino-2-cyclopenten-1-one(5-MDeaC), 3-Methyl-2-(2-carboxy-1-pyrrolidinyl)-2-cyclopentene-1-one (3-MProC), 5-Methyl-2-(2-methoxycarbonyl-1-pyrrolidinyl)-2-cyclopentene-1-one (5-MMeproC), 5-Ethyl-2-(1-pyrrolidinyl)-2-cyclopenten-1-one (5-EPC), 3,5-Dimethyl-2-(1-pyrrolidinyl)-2-cyclopenten-1-one (3,5-DMPC), 3,4-Dimethyl-2-(1-pyrrolidinyl)-2-cyclopenten-1-one (3,4-DMPC), 4,5-Dimethyl-2-(1-pyrrolidinyl)-2-cyclopenten-1-one (4,5-DMPC), 3-Methyl-2-(1-pyrrolidinyl)-2-cyclohexen-1-one (3-MPCH), 6-Methyl-2-(1-pyrrolidinyl)-2-cyclohexen-1-one (6-MPCH), 2,5-Dimethyl-4-(1-pyrrolidinyl)-3 (2H)-furanone (DMPF), 5-Methyl-4-(1-pyrrolidinyl)-3(2H)-furanone (MPF), 4,5-Dimethyl-3-(1-pyrrolidinyl)-2(5H)-furanone (2(5H)-DMPF), or 4-Methyl-3-(1-pyrrolidinyl)-2(5H)-furanone (2(5H)-MPF).

In a preferred embodiment, the cooling component includes 2,5-Dimethyl-4-(1-pyrrolidinyl)-3(2H)-furanone, 4,5-Dimethyl-3-(1-pyrrolidinyl)-2(5H)-furanone, 4-Methyl-3-(1-pyrrolidinyl)-2(5H)-furanone, or a combination thereof.

The invention relates to a food product including the consumable product described above, wherein the consumable component is edible. The consumable component includes at least one of a confectionery product, a beverage, or pet food. In a preferred embodiment, the food product includes at least one of chocolate, ice-cream, a sugar-containing confectionery product, or a malted beverage.

The invention also relates to a cosmetic product including the consumable product herein, wherein the consumable component comprises at least one of a gel, paste, cream, lotion, emulsion, or ointment in an amount sufficient for topical administration of the cosmetic product. Moreover, the invention also relates to a perfume product including the consumable product herein, wherein the consumable component includes alcohol, water, or both and the perfume product further includes a perfuming component in an amount sufficient to provide a fragrance. The invention further includes the consumable product discussed herein, wherein the consumable component includes a pharmaceutically active component and optionally at least one pharmaceutically acceptable carrier, at least one pharmaceutically acceptable excipient, or both.

The amount of the cooling component of formula (A) includes from about 0.01 mg/kg to 3000 mg/kg of the total consumable product. In one embodiment, the cooling component has a cooling threshold to odor threshold ratio that is less than that of menthol. In a preferred embodiment, the ratio is less than about 3. In one embodiment, the consumable product is substantially free of mint-odor, while in a preferred embodiment it is completely free of mint-odor.

The invention also relates to a food product formed from a consumable component and a cooling component that includes at least one of 3-Methyl-2-(1-pyrrolidinyl)-2-cyclopenten-1-one (3-MPC) or 5-Methyl-2-(1-pyrrolidinyl)-2-cyclopenten-1-one (5-MPC).

Each embodiment of the invention also relates to a method of cooling a consumer's skin or mouth by contacting a consumable component an effective amount of the cooling component to the consumer's skin or mouth to provide a cooling effect.

The invention also relates to packaging for such consumable products. In one embodiment, the cooling component and consumable component are provided in adjacent compartments separated by a removable barrier. In one preferred embodiment, the removable barrier is breakable to permit combination of the cooling component and consumable component. In another embodiment, at least a portion of one component is in a solid or powdered form and at least a portion of the other component is liquid.

### DETAILED DESCRIPTION OF THE PREFERRED EMBODIMENTS

The present invention advantageously provides a consumable product that includes a consumable component and a cooling component, the latter of which includes one or more of a group of compounds having excellent characteristics when used as an ingredient, more particularly as a cooling agent in foods, pharmaceuticals, cosmetics, or perfumes. In contrast to menthol, which exhibits a strong mint-like odor, the consumable products of the invention possess no—or at most a faint—odor and the cooling component does not substantially modify the aroma of the consumable component. The compounds in the cooling component of the invention include those of the general formula:

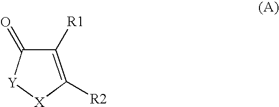

alone or in combination, particularly as an ingredient for food, cosmetic, pharmaceutical, and perfume products, wherein R1 includes N-Pyrrolidinyl, N-Pyridinyl, N-(aminodiethyl), N-(2-carboxy-pyrrolidinyl), N-(2-Methoxycarbonyl-pyrrolidinyl), or a combination thereof. R₂ includes hydrogen, methyl, or a combination thereof. X includes methylene, ethylidene, 1-Propylidene, oxy radical, or a combination thereof. Y includes methylene, ethylidene, 1-Propylidene, oxy radical, ethan-1,2-diyl, ethen-1,2-diyl, propan-1,2-diyl, ethan-1-oxy-1-yl, or a combination thereof.

In the present specification, “alone” means that only one compound of general formula (A) can be used. But, it is also possible according to the invention, to use several different types of compounds of general formula (A) in the same product.

In a first preferred embodiment, R1 includes N-Pyrrolidinyl, R₂ includes hydrogen, X includes methylene and Y includes ethylidene (5-MPC). In a second preferred embodiment, R1 includes N-Pyrrolidinyl, R₂ includes Methyl, X includes Oxy radical and Y includes methylene (MPF). In a third preferred embodiment, R₁ includes N-Pyrrolidinyl, R₂ includes methyl, X includes methylene and Y includes Oxy radical (4-methyl-3-(1-pyrrolidinyl)-2(5H)-furanone, 2(5H)MPF). In a fourth preferred embodiment of the general compound of the invention, R1 includes N-Pyrrolidinyl, R₂ includes methyl, X includes ethylidene and Y includes Oxy radical (4,5-dimethyl-3-(1-pyrrolidinyl)-2(5H)-furanone, 2(5H)DMPF). Preferred compounds of the invention include at least one of 3-Methyl-2-(1-pyrrolidinyl)-2-cyclopenten-1-one (3-MPC), 5-Methyl-2-(1-pyrrolidinyl)-2-cyclopenten-1-one (5-MPC), 3-Methyl-2-(1-piperidinyl)-2-cyclopenten-1-one (3-MPipC), 5-Methyl-2-(1-piperidinyl)-2-cyclopenten-1-one (5-MPipC), 3-Methyl-2-diethylamino-2-cyclopenten-1-one (3-MDeaC), 5-Methyl-2-diethylamino-2-cyclopenten-1-one (5-MDeaC), 3-Methyl-2-diethylamino-2-cyclopenten-1-one (3-MDeaC), 5-Methyl-2-diethylamino-2-cyclopenten-1-one (5-MDeaC), 3-Methyl-2-(2-carboxy-1-pyrrolidinyl)-2-cyclopentene-1-one (3-MProC), 5-Methyl-2-(2-methoxycarbonyl-1-pyrrolidinyl)-2-cyclopentene-1-one (5-MMeproC), 5-Ethyl-2-(1-pyrrolidinyl)-2-cyclopenten-1-one (5-EPC), 3,5-Dimethyl-2-(1-pyrrolidinyl)-2-cyclopenten-1-one (3,5-DMPC), 3,4-Di methyl-2-(1-pyrrolidinyl)-2-cyclopenten-1-one (3,4-DMPC), 4,5-Dimethyl-2-(1-pyrrolidinyl)-2-cyclopenten-1-one (4,5-DMPC), 3-Methyl-2-(1-pyrrolidinyl)-2-cyclohexen-1-one (3-MPCH), 6-Methyl-2-(1-pyrrolidinyl)-2-cyclohexen-1-one (6-MPCH), 2,5-Dimethyl-4-(1-pyrrolidinyl)-3(2H)-furanone (DMPF), 5-Methyl-4-(1-pyrrolidinyl)-3(2H)-furanone (MPF), 4,5-Dimethyl-3-(1-pyrrolidinyl)-2(5H)-furanone (2(5H)-DMPF), 4-Methyl-3-(1-pyrrolidinyl)-2(5H)-furanone (2(5H)-MPF).

The consumable product can be prepared and provided with the cooling component and consumable component already combined or in separate containers. For example, where the consumable component is a solid or powder and the cooling component is in liquid form, it might be desirable to keep them apart but preferably adjacent. A permanent or removable barrier can be provided to separate the consumable and cooling components, or the solid/powder and liquid components of the consumable product regardless of which component has the solid/powder and liquid materials, until the consumable product is ready for use. For example, a consumable food product can be prepared by having part of the consumable component in the form of milk and a cooling agent in liquid form in one compartment, and the remainder of the consumable component in the form of dry cereal in an adjacent compartment. Any of a variety of consumable components and arrangements can be provided in combination with the cooling component by those of ordinary skill in the art, particularly with reference to the disclosure herein.

The removable barrier, for example, can be broken in place or removed by a consumer so the components combine, such as by gravity or shaking an overall product container. For example, the liquid component can be in a compartment above the solid or powdered component so that the liquid can fall and mix with the solid or powdered component after the barrier between them is eliminated. In the case of a permanent barrier, one component can be added into the compartment of the other by any suitable mechanism such as a spoon, a feed line optionally connected to a pump (e.g., an atomizer to spray liquid over the solid or powdered component), or the like. By waiting to combine the consumable and cooling components of certain types of consumable products, a fresher-tasting or fresher-appearance product can be obtained. Any conventional permanent or removable barrier system can be used that is available to those of ordinary skill in the art.

In the case of a food product, the consumable component is an edible component that can include any food or edible substance suitable for consumption. By “edible” it is meant anything suitable for digestive consumption including, e.g., chewing gums and the like in addition to conventional foods that are eaten for nourishment or pleasure, or both. Preferably, these food products include anything categorized as generally recognized as safe (“GRAS”) by the FDA. Examples include beverages; confectioneries; grain-based or dough-based products including breads, crackers, cereals, pizza dough, pie crusts, pastas, and the like; fruits; vegetables; dairy-based foods including yoghurt, cheese, and cream (or any other suitable and edible form of fat whether dairy or non-dairy); meats, such as turkey, beef, pork, chicken, or fish, including ground meats, coatings or toppings for meats, and the like; pet foods or pet chew toys having an edible coating; and the like, or any suitable combination thereof. The edible component can be in the form of a liquid, powder, or solid, or a combination thereof such as a spread or paste or with distinct portions having these separate phase forms. In the case of a food product, the consumable product of the invention can be available as a ready-to-use, ready-to-eat, ready-to-bake, or raw ingredient that requires further action such as cooking or mixing with other edible substances before use by a consumer. Preferred foods include beverages, confectioneries, and pet food. More preferred beverages are malted, and more preferred confectioneries are chocolate, ice-cream, and sugar confectionery products.

In the case of a perfume product, the cooling component of the invention can be used with any suitable consumable component, such as a solvent that is preferably alcohol-based or aqueous-based. The consumable component can alternatively or additionally include any suitable perfuming ingredient or adjuvant, or a combination thereof. By “perfume product” is meant perfumes, colognes or after-shaving lotions, soaps, shower or bath gels, hygiene products, hair-care products such as shampoos or conditioners or other body or air deodorants, or the like.

The nature and diversity of the suitable perfuming ingredients or adjuvants does not need to be described in a more detailed manner here, which will anyway not be exhaustive; one of ordinary skill in the art will be able to choose such ingredients, helped by his general knowledge, according to the type of the product to be perfumed and the desired olfactory effect. Suitable perfuming ingredients belong to various chemical classes such as alcohols, aldehydes, ketones, esters, ethers, acetates, nitrites, terpenic hydrocarbons, heterocyclic compounds containing sulfur or nitrogen, and essential oils of synthetic or natural origin. Many of these ingredients are listed in reference texts such as the book of S. Arctander, Perfume and Flavor Chemicals, 1969, Montclair, N.J. USA, or its more recent versions, or in books of similar content. The proportions in which the compounds of the invention can be added to the products mentioned above vary within a large range of values. These values depend on the nature of the product to be perfumed and on the desired olfactory effect and, in a given composition where the compounds of the invention are mixed with perfuming ingredients or solvents or usual additives in perfumery, on the nature of the co-ingredients, and can be readily determined by one of ordinary skill in the art. As an example, one can cite typical concentrations of the order of about 5 weight percent to 30 weight percent, possibly more relative to the weight of the consumable product to which it is added.

In the case of a cosmetic product, the cooling component can be used with any topically applied consumable component including a cosmetic agent. The cosmetic consumable component will typically be formed as at least one of a gel, paste, cream, lotion, emulsion, or ointment, or a combination thereof, although any suitable topical formulation can be used as the consumable component. The consumable component for the cosmetic or pharmaceutical product can also include at least one of a surfactant, stabilizer, preservative, moisturizer, anti-inflammatory agent, anti-oxidant, or coloring agent.

For a pharmaceutical product, the cooling component will be used with a pharmaceutically acceptable consumable component that includes a therapeutically effective amount of a pharmaceutically active agent. The pharmaceutical product can be administered by any suitable route, although oral, intraoral, epicutaneous, transdermal, subcutaneous, intranasal, sublingual, buccal, intradural, intraocular, or nasal inhalation and like forms of administration may be employed. Preferred routes are those where the cooling component will contact a consumer's skin or mouth.

The pharmaceutical consumable component will optionally, but preferably, further include a pharmaceutically acceptable carrier, a pharmaceutically acceptable excipient component, or a combination thereof. Although any suitable excipient or carrier combination can be included, preferred excipients or carriers include starches, sugars, microcrystalline cellulose, diluents, granulating agents, lubricants, binders, disintegrating agents, and the like. Depending on the route of administration, preferred dosage forms include tablets, troches, dispersions, suspensions, solutions, capsules, patches, gel caps, syrups, elixirs, gels, powders, magmas, lozenges, ointments, creams, pastes, plasters, lotions, discs, nasal or oral sprays, aerosols, and the like. Because of their ease of administration, tablets and capsules represent the most advantageous oral dosage unit form, in which case solid pharmaceutical carriers are employed. If desired, tablets may be coated by standard aqueous or nonaqueous techniques. Preferably, administration forms for ingestion, such as tablets or capsules, would have the cooling component applied as a coating around the consumable component.

The amount of the compounds of general formula (A), alone or in combination, is from about 0.01 mg/kg to 3000 mg/kg, of the consumable product. In one embodiment, the amount of compound is from about 0.05 to 500 mg/kg, while in another embodiment, the amount of compound is from 0.1 to 50 mg/kg of the total consumable product. As already mentioned above, the above mentioned compounds of general formula (A) are used for the cooling effect they can induce to the consumable products in which they are included.

The term “about,” as used herein, should generally be understood to refer to both numbers in a range of numerals. Moreover, all numerical ranges herein should be understood to include each whole integer within the range.

The term “consumable product,” as used herein, includes any type of product that has a component that is consumed as the product is applied, administered, eaten, worn, touched, or the like, or otherwise used, and includes a cooling component according to the invention. Consumable products of the invention are typically used by any animal, preferably mammals, and more preferably humans and domesticated animals or pets. Exemplary types of consumable products include food products including chewing gum, cosmetics, perfume products, pharmaceutical products, medical devices including bandages or stitches, clothing, accessories, toothbrushes or dental floss or other dental products, tobacco-based products or tobacco-substitute products, or any other product where a cooling sensation might be of interest to a consumer. Preferred consumable products include food products, cosmetics, perfume products, and pharmaceutical products. The consumable product can be packaged in any suitable manner and as discussed herein.

“Consumable component,” as used herein, refers to the portion of a consumable product according to the invention that is at least partially consumed when the consumable product is used with a cooling component to provide a cooling sensation. Examples of such “use” of the consumable component including when the consumable product is applied, administered, eaten, worn, touched, or the like. The cooling sensation may be fleeting and designed to vanish after 1 second or less, such as upon application of a perfume, or it may be long lasting and designed to remain with the consumable product for a time as long as one hour, one day, or one week, e.g., in some types of chewing gum or on a bandage, respectively. Various consumable components are described throughout the specification and still others can be readily envisioned by those of ordinary skill in the art, particularly with reference to the discussion herein, including edible materials, dissolvable materials, materials that evaporate, and the like. For example, a consumable component may include a solvent such as water or alcohol that partly evaporates upon application to the skin, or it can include the excipients, binders, or the like used to provide flavoring and/or texture to chewing gum.

General Procedure to Synthesize Compounds of Formula (A)

An ethanolic solution (e.g., 600 mL) of a cyclic enolone compound (e.g., 100 mmol) may be refluxed in the presence of equimolar amounts (e.g., 400 mmol) of an amino compound (e.g., pyrrolidine) and acetic acid for several hours (e.g., 1-5 h). After cooling to room temperature, the solvent may be removed in vacuo and the residue may be taken up in water. The pH may be adjusted to 10 with a sodium hydroxide solution (e.g., 30% in water). The solution may then be extracted with an organic solvent (e.g. diethyl ether), the combined organic layers washed with an aqueous solution of sodium carbonate (e.g., 200 mL; 0.5 mol/L), dried over sodium sulphate and then freed from solvent in vacuo. The target compounds may further be purified by column chromatography on aluminium oxide (basic, activity III-IV, Merck, Darmstadt, Germany). Chromatography may be performed using various organic solvents in different ratios such as for example hexane (e.g., 200 ml), hexane/diethyl ether (e.g., 7:3, 400 ml), hexane/diethyl ether (e.g., 3:7, 400 ml), and diethyl ether (e.g., 400 ml). The fraction obtained with diethyl ether may be freed from solvent in vacuo affording the target compound. The compounds in Table I can be synthesized according to this general procedure.

TABLE 1
Cooling compounds of the invention synthesized

| Enolones | Amines | Target compounds |
|---|---|---|
| 2-Hydroxy-3- | Pyrrolidine | 3-Methyl-2-(1-pyrrolidinyl)-2-cyclopenten-1- |
| methyl-2- | | one (3-MPC) |
| cyclopenten-1-one | | 5-Methyl-2-(1-pyrrolidinyl)-2-cyclopenten-1- |
| | | one (5-MPC) |
| 2-Hydroxy-3- | Piperidine | 3-Methyl-2-(1-piperidinyl)-2-cyclopenten-1-one |
| methyl-2- | | (3-MPipC) |
| cyclopenten-1-one | | 5-Methyl-2-(1-piperidinyl)-2-cyclopenten-1-one |
| | | (5-MPipC) |
| 2-Hydroxy-3- | Diethylamine | 3-Methyl-2-diethylamino-2-cyclopenten-1-one |
| methyl-2- | | (3-MDeaC); 5-Methyl-2-diethylamino-2- |
| cyclopenten-1-one | | cyclopenten-1-one (5-MDeaC) |
| 2-Hydroxy-3- | L-Proline | 3-Methyl-2-(2-carboxy-1-pyrrolidinyl)-2- |
| methyl-2- | | cyclopentene-1-one (3-MProC) |
| cyclopenten-1-one | | |
| 2-Hydroxy-3- | L-Proline | 5-Methyl-2-(2-methoxycarbonyl-1- |
| methyl-2- | methylester | pyrrolidinyl)-2-cyclo-pentene-1-one (5- |
| cyclopenten-1-one | | MMeproC) |
| 2-Hydroxy-3-ethyl- | Pyrrolidine | 5-Ethyl-2-(1-pyrrolidinyl)-2-cyclopenten-1-one |
| 2-cyclopenten-1-one | | (5-EPC) |
| 2-Hydroxy-3,5- | Pyrrolidine | 3,5-Dimethyl-2-(1-pyrrolidinyl)-2-cyclopenten- |
| dimethyl-2- | | 1-one (3,5-DMPC) |
| cyclopenten-1-one | | |
| 2-Hydroxy-3,4- | Pyrrolidine | 3,4-Dimethyl-2-(1-pyrrolidinyl)-2-cyclopenten- |
| dimethyl-2- | | 1-one (3,4-DMPC); 4,5-Dimethyl-2-(1- |
| cyclopenten-1-one | | pyrrolidinyl)-2-cyclopenten-1-one (4,5-DMPC) |
| 2-Hydroxy-3- | Pyrrolidine | 3-Methyl-2-(1-pyrrolidinyl)-2-cyclohexen-1-one |
| methyl-2- | | (3-MPCH) |
| cyclohexen-1-one | | 6-Methyl-2-(1-pyrrolidinyl)-2-cyclohexen-1-one |
| | | (6-MPCH) |
| 2,5-Dimethyl-4- | Pyrrolidine | 2,5-Dimethyl-4-(1-pyrrolidinyl)-3(2H)-furanone |
| hydroxy-3(2H)- | | (DMPF) |
| furanone | | |
| 4-Hydroxy-5- | Pyrrolidine | 5-Methyl-4-(1-pyrrolidinyl)-3(2H)-furanone |
| methyl-3(2H)- | | (MPF) |
| furanone | | |
| 3-Hydroxy-4,5- | Pyrrolidine | 4,5-Dimethyl-3-(1-pyrrolidinyl)-2(5H)-furanone |
| dimethyl-2(5H)- | | (2(5H)-DMPF) |
| furanone | | |
| 3-Hydroxy-4- | Pyrrolidine | 4-Methyl-3-(1-pyrrolidinyl)-2(5H)-furanone |
| methyl-2(5H)- | | (2(5H)-MPF) |
| furanone | | |

Syntheses of 5-methyl-2-(1-pyrrolidinyl)-2-cyclopenten-1-one (5-MPC) and 3-methyl-2-(1-pyrrolidinyl)-2-cyclopenten-1-one (3-MPC)

1. From 2-hydroxy-3-methyl-2-cyclopenten-1-on (cyclotene) and pyrrolidinium acetate in ethanol A solution of cyclotene (100 mmol), pyrrolidine (400 mmol) and acetic acid (400 mmol) in ethanol (600 mL) was refluxed for 2 h. After cooling to room temperature, the solvent was removed in vacuo, the residue was taken up in water (300 mL) and the pH was adjusted to 10 with sodium hydroxide solution (30% in water). The solution was then extracted with diethyl ether (5×150 mL), the combined organic layers were washed with an aqueous solution of sodium carbonate (200 mL; 0.5 mol/L), dried over Na₂SO₄ and then freed from solvent in vacuo. The residual oil was dissolved in pentane/ethyl ether (6/4, v/v; 10 mL) and then applied onto a column (30×500 mm) filled with a slurry of aluminium oxide (basic, activity III-IV, Merck, Darmstadt, Germany) in pentane. Chromatography was performed using pentane (300 mL; fraction A), pentane/diethyl ether (9/1, v/v; 300 mL; fraction B), pentane/diethyl ether (8/2, v/v; 300 mL; fraction C), pentane/diethyl ether (7/3, v/v; 300 mL; fraction D), pentane/diethyl ether (6/4, v/v; 300 mL; fraction E), followed by pentane/diethyl ether (5/5, v/v; 300 mL, fraction F). Fraction B containing 5-MPC (1.65 g, 10% in yield) and fraction D containing 3-MPC (1.32 g, 8% in yield) were collected and freed from solvent under vacuum affording the target compounds as pale-yellow oils.

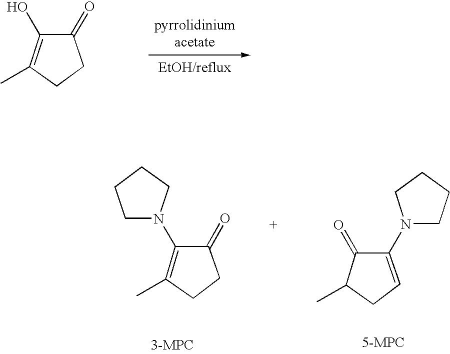

Synthetic preparation of 3-methyl-2-(1-pyrrolidinyl)-2-cyclopentene-1-one (3-MPC) and 5-methyl-2-(1-pyrrolidinyl)-2-cyclopentene-1-one (5-MPC)

2. From 2-hydroxy-3-methyl-2-cyclopenten-1-on (cyclotene) and proline dry-heated on aluminium oxide. A mixture of cyclotene (100 mmol) and proline (100 mmol) was ground with aluminium oxide (20 g, basic, activity III-IV) and then dry-heated for 10 min at 180° C. The mixture was suspended in water (100 mL) filtered, the pH was adjusted to 10 with sodium hydroxide solution (30% in water) and then extracted with diethyl ether. The work-up of the reaction mixture was performed following the procedure detailed above for the cyclotene/pyrrolidinium acetate mixture. The target compounds 5-MPC (120 mg) and 3-MPC (33 mg) were obtained as pale-yellow oils.

Spectroscopic Data:

3-MPC:

MS (EI): 165 (100; [M]+), 164 (47), 137 (34), 136 (38), 122 (53), 109 (136), 108 (43), 94 (27), 81 (26), 67 (21), 41 (27).

¹H NMR (360 MHz; CDCl₃, COSY, TOCSY): δ 1.77-1.81 (m, 2×2H, CH₂), 2.13 (s, 3H, CH₃), 2.34-2.35 (m, 2H, CH₂), 2.39-2.41 (m, 2H, CH₂), 3.40-3.44 (m, 2×2H, CH₂).

¹³C NMR (360 MHz; CDCl₃; DEPT, HMQC, HMBC): δ 17.8 [CH₃], 24.9 [2xCH₂], 30.0 [CH₂], 34.1 [CH₂], 49.5 [2xCH₂], 143.7 [C], 145.9 [C], 205.9 [CO].

5-MPC:

MS (EI): 165 (100; [M]+), 164 (32), 150 (26), 137 (22), 136 (37), 122 (87), 108 (34), 95 (34), 94 (31), 70 (21), 67 (24), 54 (24), 41 (25).

¹H NMR (360 MHz; CDCl₃, COSY, TOCSY): δ 1.16-1.18 (d, 3H, J=7.5 Hz, CH₃), 1.82-1.88 (m, 2×2H, CH₂), 2.06-2.11 (dd, J=17.7, 2.2 Hz, 1H, CHₐH), 2.35-2.43 (m, 1H, J=7.5, 2.2; CH), 2.71-2.78 (dd, J=17.7, 3.1 Hz, 1H, CHH_{b}), 3.22-3.33 (m, 2×2H, CH₂), 5.82-5.83 (t, J=3.1 Hz, 1H, CH).

¹³C NMR (360 MHz; CDCl₃; DEPT, HMQC, HMBC): δ 16.5 [CH₃], 24.8 [2xCH₂], 32.6 [CH₂], 40.2 [CH], 48.1 [2xCH₂], 123.6 [CH], 146.7 [C], 207.4 [CO].

Synthesis of 5-methyl-4-(1-pyrrolidinyl)-3(2H)-furanone (MPF)

1. From xylose and pyrrolidine. A solution of xylose (0.1 mol) and pyrrolidine (0.1 mol) in methanol (90 ml) was refluxed for 3 h, then, acetic acid (0.1 mol) was added and heating was continued for additional 2 h. After cooling, the solvent was removed in vacuo, the residue was taken up in water (100 ml), extracted with ethyl acetate (100 ml, 5 times), and the combined organic layers were extracted with aqueous 0.1M sodium hydroxide solution (3×50 ml), the organic phase was dried (sodium sulphate) and fractionated by column chromatography using aluminium oxide (basic, activity III-IV; Merck, Darmstadt, Germany) conditioned in n-hexane. Chromatography was performed using hexane (200 ml), followed by hexane/diethyl ether 7:3 (400 ml), 3:7 (400 ml), and diethyl ether (400 ml). The fraction obtained with diethyl ether was freed from solvent in vacuo affording the target compound MPF (0.9 mmol; 0.9%) as colorless oil with a purity of more than 99%.

2. From 4-hydroxy-5-methyl-3(2H)-furanone and pyrrolidine. A mixture of 4-hydroxy-5-methyl-2H-furan-3-one (10 mmol), pyrrolidine (20 mmol) and acetic acid (20 mmol) in methanol (50 ml) was refluxed for 3 h. After cooling, the solvent was removed in vacuo, the residue was taken up in H₂O (100 ml), extracted with ethyl acetate (100 ml, 5 times), and the combined organic layers were extracted with aqueous 0.1M sodium hydroxide solution (3×50 ml), the organic phase was dried (sodium sulphate) and fractionated by column chromatography as detailed above. The target compound MPF (0.5 mmol; 5% yield) was obtained as colorless oil with a purity of more than 99%.

Spectroscopic Data of MPF:

GC/MS (EI): 42 (100), 167 (95), 54 (93), 96 (76), 124 (74)

¹H NMR (360 MHz; CDCl₃, COSY, TOCSY): 1.82 (m, 2×2H, CH₂), 2.23 (s, 3H, CH₃), 3.11 (m, 2×2H, CH₂), 4.38 (s, 2H, CH₂)

¹³C-NMR (360 MHz; CDCl₃): 14.2 (CH₃), 24.7 (2xCH₂), 50.6 (2xCH₂), 72.9 (CH₂), 126.3 (C), 183.0 (C), 198.9 (CO)

Syntheses of 4,5-Dimethyl-3-(1-pyrrolidinyl)-2(5H)-furanone [2(5H)DMPF]

1. From 3-Hydroxy-4,5-dimethyl-2(5H)-furanone and pyrrolidium acetate in ethanol. A solution of 3-hydroxy-4,5-dimethyl-2(5H)-furanone (10 mmol), acetic acid (10 mmol) and pyrrolidine (10 mmol) in ethanol (50 mL) were refluxed for 3 h. After cooling to room temperature, the solvent was removed in vacuo and the residue was taken up in water (25 mL). The solution was then extracted with diethyl ether (5×10 ml), the combined organic layers were dried over sodium sulfate and then freed from solvent in vacuo. The residual oil was dissolved in pentane/diethyl ether (4/1, v/v; 5 mL) and then applied onto a column (30×500 mm) filled with a slurry of aluminium oxide (basic, activity III-IV, Merck, Darmstadt, Germany) in pentane. Chromatography was performed using pentane (300 mL; fraction A), pentane/diethyl ether (911, v/v; 400 mL; fraction B), pentane/diethyl ether (80/20, v/v; 400 mL; fraction C), pentane/diethyl ether (70/30, v/v; 400 mL; fraction D), pentane/diethyl ether (60/40, v/v; 400 mL; fraction E). Fraction E containing 2(5H)-DMPF (0.64 g, 36% in yield) was collected and freed from solvent under vacuum affording the target compounds a colorless oil.

Spectroscopic Data of 2(5H)-DMPF:

MS (E1): 181 (82; [M]+), 166 (76), 138 (28), 136 (49), 122 (100), 110 (74), 108 (93), 94 (37), 82 (36), 68 (26), 55 (43), 54 (26), 53 (24), 43 (31), 41 (44).

¹H NMR (360 MHz; CDCl₃, COSY, TOCSY): δ 1.35-1.36 (d, 3H, J=6.6 Hz, CH₃), 1.79-1.86 (m, 2×2H, CH₂), 2.03 (s, 3H, CH₃), 3.47-3.57 (m, 2×2H, CH₂), 4.66-4.72 (q, 1H, J=6.6 Hz, CH).

¹³C NMR (360 MHz; CDCl₃; DEPT, HMQC, HMBC): δ 11.8 [CH₃], 19.2 [CH₃], 24.9 [2xCH₂], 49.3 [2xCH₂], 78.0 [CH], 128.7 [C], 130.5 [C], 170.4 [CO].

Syntheses of 4-Methyl-3-(1-pyrrolidinyl)-2(5H)-furanone [2(5H)-MPF]

1. From 4-Methyl-dihydro-furan-2,3-dione and pyrrolidium acetate in ethanol. A solution of 4-methyl-dihydro-furan-2,3-dione (100 mmol, prepared according to Fleck et al., Helv. Chim. Acta 1950, 33, 130), acetic acid (100 mmol) and pyrrolidine (100 mmol) in ethanol (225 mL) was refluxed for 2,5 h. After cooling down to room temperature, the solvent was removed in vacuo and the residue was taken up in water (200 mL). The solution was then extracted with diethyl ether (5×100 ml), the combined organic layers were dried over sodium sulphate and then freed from solvent in vacuo. The residual oil was dissolved in pentane/diethyl ether (3/2, v/v; 10 mL) and then applied onto a column (30×500 mm) filled with a slurry of aluminium oxide (basic activity III-IV, Merck, Darmstadt, Germany) in pentane. Chromatography was performed using pentane (300 mL; fraction A), pentane/diethyl ether (9/1, v/v; 400 mL; fraction B), pentane/diethyl ether (80/20, v/v; 400 mL; fraction C), pentane/diethyl ether (70/30, v/v; 400 mL; fraction D), pentane/diethyl ether (60/40, v/v; 400 mL; fraction E), pentane/diethyl ether (50/50, v/v; 400 mL; fraction F). Fraction F containing 2(5H)-MPF (2.25 g, 14% in yield) was collected and freed from solvent under vacuo affording the target compound as a colorless oil.

Spectroscopic Data of 2(5H)-MPF:

MS (EI): 167 (94; [M]⁺), 166 (63), 139 (58), 138 (45), 122 (93), 120 (43), 111 (54), 110 (46), 108 (32), 95 (26), 94 (100), 82 (25), 81 (24), 80 (23), 68 (67), 67 (21), 55 (36), 54 (27), 53 (23), 41 (58). ¹H NMR (360 MHz; CDCl₃, COSY, TOCSY): δ 1.80-1.86 (m, 2×2H, CH₂), 2.09 (s, 3H, CH₃), 3.49-3.54 (m, 2×2H, CH₂), 4.53 (s, 2H, CH₂). ¹³C NMR (360 MHz; CDCl₃; DEPT, HMQC, HMBC): δ 12.4 [CH₃], 25.1 [2xCH₂], 49.5 [2xCH₂], 71.7 [CH₂], 124.3 [C], 130.7 [C], 171.6 [CO].

Sensory Analyses

Prior to sensory analysis, the purity of the synthetic taste compounds was checked by GC/MS. Determination of the cooling, as well as the aroma threshold, of the compounds was performed by trained panelists. Nasal odor thresholds (Guth, H.; Grosch, W. J. Am. Oil Chem. Soc. 1993, 70, 513-518), as well as cooling thresholds, were determined by triangle tests using tap water as the solvent. The samples were presented in order of increasing concentrations and the threshold values evaluated in three separate sessions were averaged. The values between individuals and separate sessions differed by not more than one dilution step.

The results of the sensory analyses are summarized in Table 2. Besides menthol, 5-MPC, 2(5H)-DMPF, MPF and 2(5H)-MPF had the lowest cooling threshold. Comparison of the odor threshold concentrations revealed the lowest value for menthol, which elicited a strong mint-like aroma, whereas the cooling components of the invention showed significantly higher odor thresholds. Calculating the ratio of cooling threshold to odor threshold clearly demonstrated that the compounds of formula (A), possessing no odor or only a faint odor, can be used in the cooling component and in consumable products without imparting a strong odor thereto. In comparison, for menthol the odor threshold is lower by a factor of 9.5, thereby, indicating that it is hardly possible to evoke a cooling effect in a product with menthol without having a significant mint-like odor. These data also show that by using the cooling compounds of the invention, it is now possible to evoke certain cooling effects during consumption of various products, e.g., non-mint food compositions such as, confectionery products, malted beverages, and fruity or brown flavours. Especially 2(5H)-DMPF, MPF and 2(5H)-MPF have a much lower ratio of cooling threshold to odor threshold as compared to menthol and are, therefore, very efficient cooling substances for use in or as the cooling component.

TABLE 2
Comparison of cooling and odor thresholds of selected compounds

| Cooling | Cooling | | | Ratio |
|---|---|---|---|---|
| substance | threshold^{a} | Odor threshold^{a} | Odor quality | (Cool/Odor) |
| 3-MProC | 490-735 | — | odorless | <<0.01 |
| 5-MMeproC | 112-188 | — | odorless | <<0.01 |
| DMPF | 100-140 | 30-60 | nutty, roasty | 2.7 |
| 3-MPCH | 90-150 | 45-75 | faintly mint- | 2.0 |
| | | | like | |
| 4,5-DMPC | 68-113 | 136-226 | faintly mint- | 0.5 |
| | | | like | |
| 3-MPipC | 60-100 | 80-120 | faintly | 0.8 |
| | | | amine-like | |
| 3,4-DMPC | 51-86 | 26-43 | rubber-like | 2.0 |
| 3,5-DMPC | 33-54 | 16-27 | rubber-like | 2.0 |
| 3-MPC | 29-44 | 44-73 | faintly | 0.8 |
| | | | amine-like | |
| 6-MPCH | 27-45 | 3.4-5.6 | rubber-like | 8.0 |
| 5-EPC | 27-43 | 13-22 | faintly mint- | 2.0 |
| | | | like | |
| 5-MpipC | 16-24 | 12-20 | faintly mint- | 2.7 |
| | | | like | |
| 5-MdeaC | 12-20 | 6.0-9.0 | curcuma-like | 2.1 |
| 5-MPC | 4.5-9 | 2.6-5.2 | faintly mint- | 1.7 |
| | | | like | |
| 2(5H)-DMPF | 2.0-4.0 | 32-64 | faintly mint- | 0.06 |
| | | | like | |
| MPF | 1.5-3.0 | — | odorless | <<0.01 |
| 2(5H)-MPF | 0.02-0.06 | — | odorless | <<0.01 |
| (−)-Menthol | 0.9-1.9 | 0.1-0.2 | mint-like | 9.5 |

^{a}Threshold values [mg/kg] determined in water.

In an additional experiment, the cooling thresholds of 3-MPC, 5-MPC, MPF, 2(5H)-MPF and 2(5H)-DMPF have been determined in chocolate. As given in Table 3, also in chocolate, 2(5H)-MPF was evaluated with the lowest cooling threshold of about 0.25 mg/100 g to 0.5 mg/100 g, whereas the 5-MPC showed an 8-fold higher cooling threshold. The ratio for compounds of the invention is typically less than that of menthol, preferably less than 9, more preferably less than about 3. In other embodiments, the cooling threshold to odor threshold ratio is less than about 1 or less than about 0.1.

TABLE 3
Cooling thresholds of MPF, 3-MPC and 5-MPC in milk chocolate

| Cooling compound | Cooling effect [mg/100 g chocolate] |
|---|---|
| 2(5H)-MPF | 0.25-0.5 |
| MPF | 0.8-1.5 |
| 5-MPC | 2.3-3.7 |
| 2(5H)-DMPF | 5.0-7.5 |
| 3-MPC | 38-63 |

Identification of 5-methyl-2-(1-pyrrolidinyl)-2-cyclopenten-1-one (5-MPC), 3-methyl-2-(1-pyrrolidinyl)-2-cyclopenten-1-one (3-MPC) and 2,5-dimethyl-4-(1-pyrrolidinyl)-3(2H)-furanone (DMPF) in Roasted Malt

Dark malt (50 g, Caraffa special) was frozen in liquid nitrogen and then ground in a mortar. The powder was then stirred overnight with dichloromethane (2×400 mL). The combined organic layers were then concentrated to about 50 mL in vacuo and the volatile fraction of the malt components were then isolated by high-vacuum distillation at 25° C. The distillate obtained was concentrated to about 1 mL and then fractionated by column chromatography (0.9×100 mm) on aluminium oxide (basic, activity III-IV, Merck, Darmstadt, Germany), which was conditioned in pentane. Chromatography was performed using pentane (100 mL; fraction A), pentane/diethyl ether (9/1, v/v; 100 mL; fraction B), pentane/diethyl ether (8/2, v/v; 100 mL; fraction C), pentane/diethyl ether (7/3, v/v; 100 mL; fraction D), pentane/diethyl ether (6/4, v/v; 100 mL; fraction E), pentane/diethyl ether (4/6, v/v; 100 mL; fraction F), pentane/diethyl ether (2/8, v/v; 100 mL; fraction G), followed by diethyl ether (100 mL, fraction H). Fraction B, fraction D and fraction G, respectively, were collected and analysed by GC/MS. By comparison of the retention times as well as mass spectra (El, CI) with those obtained from the synthetic reference compounds, 5-MPC (101.3 μg/Kg) could be identified in fraction B, 3-MPC (9.4 μg/Kg) in fraction D and DMPF (11.5 μg/Kg) in fraction G.

### EXAMPLES

These and other aspects of the present invention may be more fully understood with reference to the following non-limiting examples, which are merely illustrative of the preferred embodiments of the present invention, and are not to be construed as limiting the invention, the scope of which is defined by the appended claims.

### Example 1

### Application of 5-methyl-2-(1-pyrrolidinyl)-2-cyclopenten-1-one (5-MPC) and 5-methyl-4-(1-pyrrolidinyl)-3(2H)-furanone (MPF) in Mineral Water

Solutions of 10%-menthol, 10% 5-MPC and 10% MPF, respectively, in ethanol were diluted with Vittel™ mineral water. The solutions were tasted and compared with pure Vittel™ water.

| Sample | Flavor |
|---|---|
| Vittel ™ | Neutral in aroma and taste |
| 1-Menthol in Vittel ™ (10 mg/L) | Pronounced cooling effect, strong |
| | mint-like odor |
| 5-MPC in Vittel ™ (100 mg/L) | Pronounced cooling effect with a |
| | slight mint-like odor |
| MPF in Vittel ™ (400 mg/L) | Pronounced cooling effect, neutral |
| | in aroma |

### Example 2

### Application of 5-MPC and MPF in Orange Juice

Several cooling substances were evaluated in orange juice. I-Menthol contributed with a strong cooling effect at 20 mg/L. It showed also a strong mint-like aroma, thus disbalancing the overall flavour of the fruit juice. The new cooling compound 5-MPC, added at 200 mg/L, exhibited also a strong cooling effect. In addition to its cooling effect, it exhibited a weak mint-like herbal aroma note, which was however less pronounced than with 1-menthol. The addition of 1000 mg/L MPF to orange juice caused a pronounced cooling effect in the mouth cavity without any additional aroma sensation. Hence, MPF is suitable to add a cooling effect to orange juice without changing the aroma profile, contrary to 1-menthol.

### Example 3

### Application of 5-MPC and MPF in Sugar Confectionery

Sugar (100 g) was heated together with water (15 g) in a beaker on a hot plate. After the mixture became a clear solution, the sugar syrup was heated further until it became viscous due to the evaporation of water. Then 5-MPC (20 mg) and MPF (100 mg), respectively, were added. The viscous liquid was then poured into molds (3 g each) and cooled down to room temperature. The candies, which had been formed in the molds, were demolded and used for taste testing. Both candies with 5-MPC and MPF, respectively, had a pronounced cooling effect in the mouth, compared with a reference without a cooling substance according to the invention. The candies with MPF showed no additional flavour quality and were preferred to those with 5-MPC, which were slightly oily and mint-like.

### Example 4

### Application of 5-MPC in Ice Cream

Full-fat cream (250 g), milk (250 ml) and sugar (100 g) were mixed and stirred until the sugar was dissolved. Then the mixture was poured into an ice cream machine (Il Gelataio Super, Simac Inc., Gessate, Italy) and frozen within 30 minutes while stirring. In the same way, ice cream was prepared with 5-MPC (20 mg/kg). The ice cream with 5-MPC showed a pronounced long lasting cooling effect. The cool refreshing impression of this ice cream persisted much longer than with the unflavored reference compound.

### Example 5

### Topical Testing of 5-MPC, 2(5H)-DMPF, 2(5H)-MPF and MPF

Topical thresholds of cooling compounds 5-MPF, 2(5H)-DMPF, 2(5H)-MPF and MPF were determined as follows: An aliquot (0.5 mL) of a solution, containing 0.05, 0.1, 0.2, 0.5, or 1.0% of the coolant in water, was applied to a circular area (10 cm²) of the skin surface on the inside of the right forearm, midway between the wrist and the elbow, and were rubbed for 1 min. In parallel, an aliquot (0.5 mL) of pure tap water was applied as the blank onto the skin of the left forearm. After 1 min, the skin was dried. A panel of 10 subjects (male and female) were asked to rank the cooling intensity on a scale from 0 (no effect) to 5 (very strong). The values evaluated in three different sessions at two days were averaged. The values between individuals and separate sessions differed not more than 2 scores.

Topical testing of 5-MPC and MPF on the inside of the forearm

| | Cooling intensity of^{a} | | | |
|---|---|---|---|---|
| Concentration [%] | 2(5H)-MPF | 2(5H)-DMPF | MPF | 5-MPC |
| 0.00020 | 0 | | 0 | 0 |
| 0.00039 | 1 | | 0 | 0 |
| 0.00078 | 2 | | 0 | 0 |
| 0.00156 | 3 | | 0 | 0 |
| 0.00313 | 5 | | 0 | 0 |
| 0.00625 | 5 | 0 | 0 | 0 |
| 0.0125 | n.d. | 1 | 0 | 0 |
| 0.025 | n.d. | 3 | 0 | 0 |
| 0.05 | n.d. | 5 | 1 | 0 |
| 0.1 | n.d. | 5 | 2 | 0 |
| 0.2 | n.d. | 5 | 4 | 1 |
| 0.5 | n.d. | 5 | 5 | 3 |
| 1.0 | n.d. | 5 | 5 | 5 |

^{a}Cooling effect on skin was determined in tap water by using a topical test scoring the cooling intensity on a scale from 0 (no effect) to 5 (very strong).
n.d. means cooling effect not detected.

Although preferred embodiments of the invention have been illustrated in the accompanying drawings and described in the foregoing Detailed Description, it will be understood that the invention is not limited to the embodiments disclosed, but is capable of numerous rearrangements and modifications of parts and elements without departing from the spirit of the invention. It will be understood that the mechanical and chemical details of every embodiment may be slightly different or modified by one of ordinary skill in the art without departing from the present invention.

What is claimed is:

1. A consumable product that comprises:
a consumable component; and
a cooling component present in an amount sufficient to provide a cooling sensation to a consumer, which component comprises one or more compounds each having the general formula: 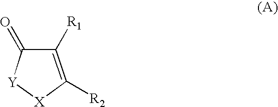
wherein R₁ is selected from the group consisting of N-Pyrrolidinyl, N-Pyridinyl, N-(aminodiethyl), N-(2-carboxy-pyrrolidinyl), piperidinyl, or N-(2-Methoxycarbonyl-pyrrolidinyl); R₂ is selected from the group consisting of hydrogen or methyl; X is selected from the group consisting of methylene, ethylidene, 1-Propylidene, or oxy radical; and Y is selected from the group consisting of methylene, ethylidene, 1-propylidene, oxy radical, ethan-1,2-diyl, ethen-1,2-diyl, propan-1,2-diyl, or ethan-1-oxy-1-yl, provided that when R₁ is N-pyrrolidinyl, X is methylene, and Y is ethylidene then R₂ cannot be hydrogen, and provided that when R1 is N-pyrrolidinyl, and X and Y are each methylene, then R₂ cannot be methyl.

2. The consumable product of claim 1, wherein R₁ is N-pyrrolidinyl, R₂ is methyl, X is oxy radical and Y is methylene.

3. The consumable product of claim 1, wherein R₁ is N-Pyrrolidinyl, R₂ is methyl, X is methylene and Y is oxy radical.

4. The consumable product of claim 1, wherein R₁ is N-Pyrrolidinyl, R₂ is methyl, X is ethylidene and Y is oxy radical.

5. The consumable product of claim 1, wherein the cooling component comprises at least one of 3-Methyl-2-(1-pyrrolidinyl)-2-cyclopenten-1-one (3-MPC), 5-Methyl-2-(1-pyrrolidinyl)-2-cyclopenten-1-one (5-MPC), 3-Methyl-2-(1-piperidinyl)-2-cyclopenten-1-one (3-MPipC), 5-Methyl-2-(1-piperidinyl)-2-cyclopenten-1-one (5-MPipC), 3-Methyl-2-diethylamino-2-cyclopenten-1-one (3-MDeaC), 5-Methyl-2-diethylamino-2-cyclopenten-1-one (5-MDeaC), 3-Methyl-2-diethylamino-2-cyclopenten-1-one (3-MDeaC), 5-Methyl-2-diethylamino-2-cyclopenten-1-one(5-MDeaC), 3-Methyl-2-(2-carboxy-1-pyrrolidinyl)-2-cyclopentene-1-one (3-MProC), 5-Methyl-2-(2-methoxycarbonyl-1-pyrrolidinyl)-2-cyclo-pentene-1-one (5-MMeproC), 5-Ethyl-2-(1-pyrrolidinyl)-2-cyclopenten-1-one (5-EPC), 3,5-Dimethyl-2-(1-pyrrolidinyl)-2-cyclopenten-1-one (3,5-DMPC), 3,4-Dimethyl-2-(1-pyrrolidinyl)-2-cyclopenten-1-one (3,4-DMPC), 4,5-Dimethyl-2-(1-pyrrolidinyl)-2-cyclopenten-1-one (4,5-DMPC), 3-Methyl-2-(1-pyrrolidinyl)-2-cyclohexen-1-one (3-MPCH), 6-Methyl-2-(1-pyrrolidinyl)-2-cyclohexen-1-one (6-MPCH), 2,5-Dimethyl-4-(1-pyrrolidinyl)-3(2H)-furanone (DMPF), 5-Methyl-4-(1-pyrrolidinyl)-3 (2H)-furanone (MPF), 4,5-Dimethyl-3-(1-pyrrolidinyl)-2(5H)-furanone (2(5H)-DMPF), or 4-Methyl-3-(1-pyrrolidinyl)-2(5H)-furanone (2(5H)-MPF).

6. The consumable product of claim 1, wherein the cooling component comprises 2,5-Dimethyl-4-(1-pyrrolidinyl)-3(2H)-furanone, 4,5-Dimethyl-3-(1-pyrrolidinyl)-2(5H)-furanone, 4-Methyl-3-(1-pyrrolidinyl)-2(5H)-furanone, or a combination thereof.

7. A food product comprising the consumable product of claim 1, wherein the consumable component is edible.

8. The food product of claim 7, wherein the consumable component comprises at least one of a confectionery product, a beverage, or pet food.

9. The food product of claim 8, comprising at least one of chocolate, ice-cream, a sugar-containing confectionery product, or a malted beverage.

10. A cosmetic product comprising the consumable product of claim 1, wherein the consumable component comprises at least one of a gel, paste, cream, lotion, emulsion, or ointment in an amount sufficient for topical administration of the cosmetic product.

11. A perfume product comprising the consumable product of claim 1, wherein the consumable component comprises alcohol, water, or both and the perfume product further comprises a perfuming component in an amount sufficient to provide a fragrance.

12. A pharmaceutical composition comprising the consumable product of claim 1, wherein the consumable component comprises a pharmaceutically active component and optionally at least one pharmaceutically acceptable carrier, at least one pharmaceutically acceptable excipient, or both.

13. The consumable product of claim 1, wherein the amount of the cooling component of formula (A) comprises from about 0.01 mg/kg to 3000 mg/kg of the total consumable product.

14. The consumable product of claim 1, wherein the cooling component has a cooling threshold to odor threshold ratio that is less than that of menthol.

15. The cooling component of claim 14, wherein the ratio is less than about 3.

16. The consumable product of claim 13, wherein the consumable product is completely free of mint-odor.

17. The food product of claim 7 comprising at least one of 3-Methyl-2-(1-pyrrolidin yl)-2-cyclopenten-1-one (3-MPC) or 5-Methyl-2-(1-pyrrolidinyl)-2-cyclopenten-1-one (5-MPC).

18. The consumable product of claim 1, wherein the cooling component and consumable component are provided in adjacent compartments separated by a removable barrier.

19. The consumable product of claim 18, wherein the removable barrier is breakable to permit combination of the cooling component and consumable component.

20. The consumable product of claim 1, wherein at least a portion of one component is in a solid or powdered form and at least a portion of the other component is liquid.
